(19) United States
(12) Patent Application Publication
(10) Pub. No.: US 2017/0106068 A1
(43) Pub. Date: Apr. 20, 2017

(54) ORAL COMPOSITION AND METHODS FOR IMMUNOTHERAPY

(71) Applicants: Aldar BOURINBAIAR, College Park, MD (US); Vichai JIRATHITIKAL, Vancouver (CA)

(72) Inventors: Aldar BOURINBAIAR, College Park, MD (US); Vichai JIRATHITIKAL, Vancouver (CA)

(21) Appl. No.: 15/318,279

(22) PCT Filed: Jun. 15, 2015
(86) PCT No.: PCT/US15/35745

§ 371 Date: Dec. 12, 2016

Related U.S. Application Data
(60) Provisional application No. 61/997,902, filed on Jun. 13, 2014.

(51) Int. Cl. A61K 39/00; A61K 9/00

(52) U.S. Cl. CPC A61K 39/0011; A61K 9/0053; A61K 2039/542; A61K 2039/60

(57) ABSTRACT

A composition includes a metal chemically bound to at least one heat-denatured tumor antigen and at least one heat-denatured alloantigen. The tumor antigen and/or the alloantigen are hydrolyzed. The composition can be formulated in a tablet or pill. Methods of treatment of cancer and inflammatory diseases are also provided by administering, e.g., orally, the composition to a subject in need thereof.

### CROSS REFERENCE TO RELATED APPLICATION

This application claims priority to U.S. Provisional Application No. 61/997,902, filed Jun. 13, 2014, the disclosure of which is incorporated herein by reference in its entirety.

### FIELD OF THE INVENTION

The present invention belongs to the field of oncology and immunology and broadly relates to an oral composition and methods for immunotherapy of malignant and autoimmune diseases.

### BACKGROUND

In 2012, there were estimated 14.1 million new cancer cases worldwide, 8.2 million cancer deaths and 32.6 million people living with cancer. Cancer affects almost every organ of the human body. Despite popular belief, less than five percent of cancer is genetically inherited. Instead there is an emerging link between inflammation and cancer (see for example review by Lu H, Ouyang W, Huang C. Inflammation, a key event in cancer development. Mol Cancer Res 2006, 4:221-33). By way of example, there is a liver cancer, of which most are of particular type called hepatocellular carcinoma (HCC)—also known to be an inflammatory disease (Bishayee A. The inflammation and liver cancer. Adv Exp Med Biol 2014, 816:401-35, the abstract of which is incorporated herein by way of reference). HCC is the fifth most common cancer worldwide and ranks third in fatality rate. About one million new cases are diagnosed every year with almost an equal number of deaths—highlighting the unmet need for better treatments. The conventional interventions such as surgery, radiation and chemotherapy have severe toxic effects and are generally not very effective. The attention is now shifted to biological treatments, i.e., immunotherapy.

Cancer immunotherapy relies on use of the immune system of the host to prevent or eliminate the cancer. There are three main groups of immunotherapies: cell-based therapies, antibody therapies and cytokine therapies. They all exploit the fact that cancer cells often have subtly different molecules on their surface that can be detected by the immune system. These molecules, known as cancer or tumor antigens, are most commonly proteins but also include other molecules such as carbohydrates. According to conventional thought immunotherapy provokes the immune system into attacking the tumor cells by using these tumor antigens as targets.

Cell-based therapies, more commonly known as cancer vaccines, usually involve the removal of immune cells from someone with cancer, e.g., from the blood. Immune cells specific for the tumor will be activated, grown in vitro and injected back to the person with cancer where the immune cells provide the immune response against the cancer. Cell types that can be used in this way are natural killer cells, lymphokine-activated killer cells, cytotoxic T cells and dendritic cells. The first clinically approved cell-based therapy is Dendreon's Provenge (Sipuleucel) vaccine, which is used for the treatment of prostate cancer.

Antibody therapies are currently the most popular form of immunotherapy, with many approved treatments for a wide range of cancers. Antibodies are proteins produced by the immune system that bind to a target antigen on the surface of a tumor cell. In normal physiology they are used by the immune system to fight pathogens. Each antibody is specific to one or a few proteins and those that bind to cancer antigens are used in the treatment of cancer. Interventions using antibodies against CD47, GD2 ganglioside carbohydrate antigen, immune checkpoint programmed cell death 1 protein (PD-1; also known as CD279) and its ligand, PD-1 ligand 1 (PD-L1), and EGF receptor are most intensively investigated. There are several antibodies currently approved for the treatment of cancer, e.g., Alemtuzumab, Bevacizumab, Brentuximab, Cetuximab, Gemtuzumab, Ibritumomab, Ipilimumab, Ofatumumab, Panitumumab, Rituximab, Tositumomab and Trastuzumab.

Interferon-α and Interleukin-2 (IL-2) are examples of cytokines, proteins that regulate and coordinate the behavior of the immune system. They have the ability to enhance the anti-tumor activity of the immune system and thus can be used as treatments in cancer. Interferon-α is used in the treatment of hairy-cell leukemia, AIDS-related Kaposi's sarcoma, follicular lymphoma, chronic myeloid leukemia and malignant melanoma. IL-2 is used in the treatment of malignant melanoma and renal cell carcinoma.

In addition, certain compounds, primarily polysaccharide compounds, can up-regulate the immune system and may have anti-cancer properties. For example, beta-glucans, such as lentinan and other polysaccharides from edible fungi have been tested for their anti-cancer potential (e.g., U.S. Pat. No. 7,011,845). These compounds are usually given orally as opposed to other immunotherapies which are given by injection.

Increasing number of studies is now devoted to development of various cancer immunotherapies. Several approaches have been proposed, including Picibanil (OK-432)—a mixture of Streptococcus antigens (U.S. Pat. No. 5,559,211); heat shock proteins, e.g., HSP70 (U.S. Pat. Nos. 8,729,111 and 6,139,841); oncolytic viruses (U.S. Pat. No. 8,450,106); DNA vaccines and gene therapy (U.S. Pat. Nos. 8,216,595 and 5,631,236); various means of so-called adoptive cell transfer including dendritic cells (US Patent Application Publication Nos. US2011/0076290 and US2005/0260227), lymphokine-activated killer cells (U.S. Pat. No. 8,691,568), cytokine- or chemokine-induced killer cells (U.S. Pat. Nos. 6,716,425 and 6,562,347), natural killer or NK cells (U.S. Pat. No. 8,450,112); native tumor cells alone or mixed with additional stimulants (U.S. Pat. No. 6,207,147); tumor antigens alone or in combination with an allogeneic antigen (U.S. Pat. No. 7,438,922); tumor lysates in various forms (U.S. Pat. No. 4,108,983); single and multiple cytokines; e.g., IL-2 and GM-CSF (U.S. Pat. No. 5,478,556); immune adjuvants of various origin (U.S. Pat. No. 8,216,595); monoclonal antibodies (U.S. Pat. No. 8,680,247) and various mixtures thereof (U.S. Pat. Nos. 5,126,132 and 7,919,079). The disclosure of above cited patents is incorporated herein by way of reference. There is a vast array of cancer immunotherapies proposed by experts in the field. When these immunotherapies were tested in clinical trials, the instances of complete remissions of cancer (i.e., cure) occurred rarely; most have shown modest effect on survival, typically just a few months. Thus, despite considerable effort in the field and tremendous advances in the understanding of the immunology of cancer, it is clear that more effective immunotherapies need to be developed.

### SUMMARY OF THE INVENTION

In one aspect, the present invention provides an oral composition comprising a metal bound to at least one tumor antigen and at least one alloantigen or fragments thereof.

In preferred embodiments, at least one tumor antigen and at least one alloantigen is hydrolyzed.

In certain embodiments, at least one tumor antigen and at least one alloantigen is heat-denatured.

In one embodiment, the metal is magnesium. In another embodiment, the metal is calcium.

The tumor antigen can be a protein, a peptide, a hapten, a polysaccharide, a glycoprotein, a lipopolysaccharide, or a DNA molecule.

In some embodiments, the tumor antigen is selected from the group consisting of AFP, CEA, CD31, CD34, CD99, CD117, GCDFP-15, EMA, ETA, MPG, p97, Neu, c-myc, raf, ras, MAGE, BAGE, DAGE/Prame, GAGE, RAGE SMAGE, NAG, CQA 72/4, Laminin-P1, Yale Col. Sr. Factor, UGP, hCG, PD1 (CD279), PTPRC (CD45), HMB-45, MART-1/Melan-A, Myo D1, MSA, M2-PK, PLAP, PSA, gp100, MUC-1, MUC-2, MUC16, TRP-1, MUM-1, CDK-4, TAG-72, CA-15-3, CA-19-9, CA-27-29, CA-72-4, CA-125, Cyfra 21-1, CYP24, NSE, AMFr, M-344, 19a21 1, erb-2, p15, p21, p53, Bcr/Abl breakpoint peptide, WT1, HER-2/neu, PD-41, TCSF, GA733-2, HPV16 E7, E6, MZ2-E, B7.1, B7.2, HOM-MEL-40, HOM-MEL-55, NY-COL-2, HOM-HD-397, HOM-RCC-1.14, HOM-HD-21, HOM-NSCLC-11, HOM-MEL-2.4, HOM-TES-11, GRP78, EGFR, BRCA1, BRCA2, APC, HER2, PSA, NY-ESO-1, 4-5, PSMA, PSCA, EpCam, POA, GnT-V, TERT, calcitonin, calretinin, chromogranin, cytokeratin, desmin, inhibin, keratin, recoverin, kallikrein, beta-catenin, annexin, mammoglobin, tyrosinase and mixtures thereof.

In some embodiment, the tumor antigen is an antigen derived from a cancer cell, said cancer selected from the group consisting of Adrenal cancer, Anal cancer, Bile Duct cancer, Bladder cancer, Bone cancer, Brain/CNS tumors, Breast cancer, Castleman disease, Cervical cancer, Colon/Rectum cancer, Endometrial cancer, Esophagus cancer, Ewing Tumor, Eye cancer, Gallbladder cancer, Gastric cancer, Gastrointestinal carcinoid tumors, Gastrointestinal Stromal Tumor, Gestational Trophoblastic disease, Hodgkin disease, Kaposi sarcoma, Laryngeal and Hypopharyngeal cancer, Leukemias, e.g., ALL, AML, CLL, CML, and CMML, Lymphoma, Non-Hodgkin lymphoma, Liver cancer, Lung cancer, Malignant mesothelioma, Multiple myeloma, Myelodysplastic syndrome, Nasal cavity and Paranasal sinus cancer, Nasopharyngeal cancer, Neuroblastoma, Oral cavity and Oropharyngeal cancer, Osteosarcoma, Ovarian cancer, Pancreatic cancer, Penile cancer, Pituitary tumor, Prostate cancer, Renal cancer, Retinoblastoma, Rhabdomyosarcoma, Salivary gland cancer, Sarcoma, Basal and squamous cell skin cancer, Melanoma, Merkel cell cancer, Small intestine cancer, Stomach cancer, Testicular cancer, Thymus cancer, Thyroid cancer, Uterine sarcoma, Vaginal cancer, Vulvar cancer, Waldenstrom macroglobulinemia, and Wilms tumor.

In one embodiment, the tumor antigen is AFP.

In one embodiment, the alloantigen is albumin.

In some embodiments, the weight ratio between the tumor antigen and the alloantigen is anywhere between 1:1 to 1:1,000,000, or between 1:50 and 1:200.

In another aspect, the present invention provides a composition comprising a metal bound to at least one heat-denatured, hydrolyzed alloantigen and at least one heat-denatured, hydrolyzed tumor antigen, said composition formulated as a pill. The metal can be magnesium or calcium or any other suitable metal capable of forming organometallic bond. The hydrolyzed and denatured tumor antigen and hydrolyzed and denatured alloantigen can be a peptide, such as an oligopeptide, or a polypeptide.

In another aspect, the present invention provides a method for treating a cancer in a subject in need thereof, comprising orally administering to the subject a therapeutically effective dose of a composition disclosed herein.

In another aspect, the present invention provides a method of inducing an anti-inflammatory immune reaction in a subject. The method includes orally administering a therapeutically effective dose of a composition to the subject, the composition comprising a tumor antigen and an alloantigen bound to a metal. In some embodiments, the tumor antigen and alloantigen are hydrolyzed. In other embodiments, wherein the tumor antigen and alloantigen are heat-denatured.

In yet another aspect, the present invention provides a method of preparing a composition, which includes: obtaining a mixture of a tumor antigen and an alloantigen; denaturing the tumor antigen and the alloantigen; and forming at least one of a metal-bound denatured tumor antigen and a metal-bound denatured alloantigen. The method can further comprise hydrolyzing at least one of the tumor antigen and the alloantigen.

In some embodiments, the tumor antigen is obtained from a pooled blood of donors diagnosed with desired types of cancer. In other embodiments, the tumor antigen is obtained from cancer cell lines or tissues.

In some embodiments, the alloantigen is derived from non-malignant cells derived from the peripheral blood or cell lines.

In some embodiments, the denaturing step comprises applying heat.

### BRIEF DESCRIPTION OF THE DRAWINGS

FIG. 1 shows computed tomography (CT) scans of a patient having hepatocellular carcinoma before and after immunotherapy treatment with a composition of the present invention. Panel (a) shows single large tumor in the right lobe of liver before tumor was surgically removed; (b) after four months multiple malignant lesions recurred in the left lobe; the AFP levels at that time were 92,407 IU/ml; (c) all lesions were cleared after 8 months of daily single dose of the composition; AFP levels at this timepoint came down to below normal threshold, 2.3 IU/ml. Patient is now healthy and doing very well 2 years after treatment.

FIG. 2 shows representative effect of 48-hour in vitro incubation of T lymphocytes with a vaccine composition of the present invention (10⁻⁶ dilution) on expression of IFN-γ, TNF-α; IL-2 and cell proliferation/activation markers, i.e., Ki-67 and CD69 as shown in lower row. The upper row represents control unstimulated T cells as analyzed by flow cytometry at the same time. The data show that expression IFN-γ increased from 1.65% to 9.11%; TNF-α decreased from 5.05% to 0.38%; IL-2 from 1.65% to 1.45%; Ki-67 from 10.7% to 44.3%; and CD69 from 10.5% to 16.1%.

### DETAILED DESCRIPTION

The present invention provides a vaccine composition (also referred to herein as a vaccine, a therapeutic vaccine or immunotherapy composition), and methods of making and using the same for treating and/or preventing cancer and certain inflammatory diseases.

An understanding of the mechanism is not necessary to practice the present invention and the present invention is not limited to any particular mechanism of action. Nevertheless, the clinical experience with the instant composition in several types of cancer and inflammatory diseases suggests that this invention goes against several dogmas currently prevailing in the field.

First, the present invention goes against the consensus that it is important to preserve antigenic epitopes in their native form so that they can be properly recognized by a subject's immune system. The term “epitope” stands for an antigen or a fragment thereof as an immunogenic determinant capable of specific binding to an antibody or a T-cell receptor. An epitope usually consists of chemically active surface groupings of molecules such as amino acids or sugar side chains, and usually have specific three dimensional structural characteristics, as well as specific charge characteristics. The instant composition includes denatured and hydrolyzed antigens which have irreversibly lost their native configuration yet surprisingly are highly effective in immunotherapies for cancer and autoimmune diseases.

The term “immune tolerance” does not imply immune suppression or anergy, rather it is an active immune process of anti-inflammatory nature, which is as potent as classical immune activation. Current consensus holds a view that cancer arises from host's “immune tolerance” and resulting failure of the immune system to fight off cancer (U.S. Pat. No. 5,723,718).

Third, the present invention requires no immune adjuvant or immunostimulant to enhance the immune reaction of the host.

Fourth, the present invention utilizes allogeneic antigens (alloantigens) along with tumor antigens to improve the efficacy of the composition.

Fifth, gut cells which line epithelial surface of the intestine where antigen is absorbed serve as antigen presenting cells much more efficiently than dendritic cells which are believed to be the critical cells for many cancer vaccines proposed by others. But so far dendritic cell vaccines have failed to cure anyone and clinical responses were usually seen in no more than 15% of cases.

Sixth, it has been discovered that the instant composition made from blood of patients with cancer is equally effective as one made from tumor tissues or cells. This phenomenon is perhaps due to the fact that literally millions of cancer cells develop spontaneously in the human body every day but in most cases they do not cause disease as they are held in check by the immune system.

Finally, the instant composition is in oral form and this makes dramatic difference from prior art cancer vaccines, which are made in injectable form. When a vaccine is given by injection one needs to have highly purified tumor antigen since impurities can produce undesired adverse reactions—phenomenon well known in the prior art, which indicates that multivalent vaccines have more adverse reactions than monovalent single epitope/antigen vaccines. But at the same time a vaccine with fewer number of antigens is less effective. Thus a dilemma that was facing artisans has been solved by making the instant vaccine oral. Giving mixture of antigens without separating antigens but giving them as a pooled mixture does not cause any adverse reaction since it is given orally and host's mucosal immune system handles them in a similar way as it handles the digestion of food which has a multitude of foreign antigens. Unlike systemic immunity, the mucosal immune system has been specialized and highly adapted to handle a vast antigenic diversity.

In the present disclosure, the terms “cancer,” “neoplasm,” “malignancy,” and “tumor” are used interchangeably and in either the singular or plural form. These terms refer to cells that have undergone a malignant transformation that makes them pathological to the host organism and ending with fatal outcome. Several cancer types are within the scope of instant invention, which include but are but not limited to Adrenal Cancer, Anal Cancer, Bile Duct Cancer, Bladder Cancer, Bone Cancer, Brain/CNS Tumors, Breast Cancer, Castleman Disease, Cervical Cancer, Colon/Rectum Cancer, Endometrial Cancer, Esophagus Cancer, Ewing Tumor, Eye Cancer, Gallbladder Cancer, Gastric Cancer, Gastrointestinal Carcinoid Tumors, Gastrointestinal Stromal Tumor (GIST), Gestational Trophoblastic Disease, Hodgkin Disease, Kaposi Sarcoma, Kidney Cancer, Laryngeal and Hypopharyngeal Cancer, many types of Leukemia, e.g., ALL, AML, CLL, CML, and CMML, Lymphoma, Non-Hodgkin Lymphoma, Liver Cancer, Lung Cancer, Malignant Mesothelioma, Multiple Myeloma, Myelodysplastic Syndrome, Nasal Cavity and Paranasal Sinus Cancer, Nasopharyngeal Cancer, Neuroblastoma, Oral Cavity and Oropharyngeal Cancer, Osteosarcoma, Ovarian Cancer, Pancreatic Cancer, Penile Cancer, Pituitary Tumor, Prostate Cancer, Renal Cancer, Rhabdomyosarcoma, Retinoblastoma, Salivary Gland Cancer, Sarcoma, Basal and Squamous Cell Skin Cancer, Melanoma, Merkel Cell cancer, Small Intestine Cancer, Stomach Cancer, Testicular Cancer, Thymus Cancer, Thyroid Cancer, Uterine Sarcoma, Vaginal Cancer, Vulvar Cancer, Waldenstrom Macroglobulinemia, and Wilms Tumor among many others. When referring to a type of cancer that normally manifests as a solid tumor, a “clinically detectable” tumor is one that is detectable on the basis of tumor mass; e.g., by CT scan, magnetic resonance imaging (MRI), X-ray, ultrasound or palpation. In cancer of blood, i.e., leukemia, other diagnostic criteria are used by testing for example blood samples for abnormalities.

The term “protein” as used herein refers to functionally active biological molecules, consisting of one or more amino acid residues. Peptides, oligopeptides, polypeptides and proteins are terms used to describe amino acid strings of various lengths from low to high weight end. The term “oligopeptide” as used herein refers to peptides that contain approximately 10 to 30 amino acids. Although there is not a firm cutoff, for the sake of convenience, the term “polypeptide” as used herein would refer to a peptide segment having a length of over 30 amino acids.

As used herein, the term “effective amount” or “therapeutically effective amount” refers to a dose of the instant vaccine required (e.g., when administered to a subject) to generate a desired immune response in the subject.

As used hereinafter the term “excipient” refers to a substance formulated alongside the active ingredient (API) of a medication such as pill, capsule or tablet. Excipients are usually necessary to help make the oral dosage form, such as a pill or tablet, stable and usable. The selection of appropriate pharmaceutically acceptable excipients is well known to those skilled in the art. Generally excipients comprise antiadherents such as magnesium stearate; binders: saccharides and their derivatives, disaccharides: sucrose, lactose; polysaccharides and their derivatives: starches, cellulose or modified cellulose such as microcrystalline cellulose and cellulose ethers such as hydroxypropyl cellulose; sugar alcohols such as xylitol, sorbitol or maltitol; protein: gelatin; synthetic polymers: polyvinylpyrrolidone, polyethylene glycol; coating ingredients: hydroxypropyl or methylcellulose film coating, shellac, corn protein zein or other polysaccharides, fatty acids, waxes, plastics, and plant fibers; disintegrants such as crosslinked polymers: polyvinylpyrrolidone, carboxymethyl cellulose (croscarmellose sodium), starch glycolate; fillers such as cellulose calcium phosphate, lactose, sucrose, glucose, mannitol, sorbitol, calcium carbonate, and magnesium stearate; flavours, e.g. fruit extract or artificial flavours like mint, cherry or anise; various colours, e.g., titanium oxide, rose ponceau, etc.; lubricants like talc or silica, and fats, e.g. vegetable stearin, magnesium stearate or stearic acid; glidants including fumed silica, talc, and magnesium carbonate; sorbents; preservatives such as antioxidants: vitamin A, vitamin E, vitamin C, retinyl palmitate, selenium, cysteine, methionine, citric acid, sodium citrate, methyl paraben, propyl paraben; sweeteners and alike.

Non-limiting examples of metals used in this disclosure include aluminum, antimony, boron, chromium, copper, gold, iron, lead, lithium, sodium, calcium, potassium, magnesium, manganese, platinum, selenium, silicon, sodium, silver, titanium, strontium, tin, tungsten, vanadium and zinc. More preferable are metals and salts thereof, which are part of the daily diet such as sodium, potassium, calcium, manganese and magnesium, although other equally suitable common metals in the diet include iron, cobalt, copper, zinc, molybdenum, iodine, and selenium. Even more preferable are magnesium and calcium which are two most abundant divalent cations in serum. Non-preferred metals are toxic or heavy metals like arsenic, beryllium, cadmium, chromium, lead, mercury and platinum. In preferred embodiments, the metals can be magnesium or calcium. In one embodiment the metal is magnesium. The metal can be bound to the active ingredients (API), the tumor antigens and the alloantigens, to form insoluble precipitate or aggregate.

Non-limiting examples of salts for the presently disclosed subject matter include, but are not limited to: acetate, adipate, alginate, aspartate, benzenesulfonate, benzoate, bisulfate, bromide, butyrate, camphorate, camphorsulfonate, chloride, citrate, cyclopentanepropionate, digluconate, dodecylsulfate, ethanesulfonate, flucoheptanoate, fumarate, glycerophosphate, hemisulfate, heptanoate, hexanoate, iodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, organometallic salt and the like. For therapeutic use, salts of the compounds of the present invention are contemplated as being pharmaceutically acceptable. However, salts of acids and bases that are non-pharmaceutically acceptable or serve as inactive ingredients may also find use, for example, in the initial steps of preparation of a pharmaceutically acceptable compound or as an excipient.

As used herein a “metal salt” is a compound which is produced as a result of chemical reaction of a metal in contact with inorganic or organic acids or bases and physiologically tolerated in the target subject. Examples of acids include, but are not limited to acetic, benzenesulfonic acid, benzoic, citric, ethanesulfonic, formic, fumaric, glycolic, hydrobromic, hydrochloric, lactic, maleic, malonic, methanesulfonic, naphthalene-2-sulfonic, nitric, perchloric, phosphoric, salicylic, succinic, sulfonic, sulfuric, tartaric, toluene-p-sulfonic, and the like. Examples of bases include, but are not limited to alkali metal (e.g., sodium) hydroxide, alkaline earth metal (e.g., magnesium) hydroxides, ammonia, and the like. Without limiting to metal salts having acid and alkali bonds, other metal salts are also suitable such as organometallic compounds containing bond between an organic ligand, i.e., carbon, oxygen or nitrogen and a metal.

In one aspect, the present invention provides a composition (also referred to as a vaccine, a vaccine composition, or immunotherapy composition) which comprises a denatured tumor antigen or fragments thereof, a denatured alloantigen or fragments thereof, and a metal bound to the above components. The composition can further comprise pharmaceutically acceptable excipients for an oral dosage form.

As used herein, a “tumor antigen” refers to an antigenic or immunogenic substance produced by tumor cells, i.e., it can trigger an immune response in the host. However, the tumor antigen can be as a single antigen or have multiple components. Most often the antigen is a protein, but other forms of tumor antigen are also included for purpose of the instant invention. A tumor antigen can be a protein (including a recombinant protein), a peptide, a hapten, a polysaccharide, a glycoprotein, a lipopolysaccharide, a DNA molecule, or mixtures thereof. As used herein the term “recombinant protein” refers to a protein that is produced by expression of recombinant DNA; the term “peptide’ refers to a fragment of a protein composed of amino acids; the term “fragment” when in reference to a protein refers to a size anywhere from two amino acid residues to the entire amino acid sequence of the protein minus one amino acid; the term “hapten” refers to a small molecule that can elicit an immune response when attached to a large carrier such as a protein; the term “polysaccharide” refers to a carbohydrate polymer composed of long chains of monosaccharide units bound together by glycosidic linkages, well known example of polysaccharide is beta-glucan; the term “glycoprotein” refers to a protein that contains oligosaccharide chains covalently attached to a polypeptide (an example of a glycoprotein is tumor-associated glycoprotein 72 (TAG-72) which is found on the surface of many cancer cells, including ovary, breast, colon, and pancreatic cells; the term “lipopolysaccharide” (also known as lipoglycan or endotoxin) refers to a large molecule consisting of lipid and polysaccharide; the term “DNA molecule” refers to a polynucleotide encoding a protein; the term “cancer cell” is an abnormal cell dividing without control, a plurality of which may form tumor masses or circulate in the blood.

Example tumor antigens for purpose of this disclose include oncofetal antigens such as alpha fetoprotein (AFP) and carcinoembryonic antigen (CEA), melanoma MPG and p97, carcinoma Neu oncogene product, members of the MAGE family, the BAGE family, the DAGE/Prame family, the GAGE family, the RAGE family, the SMAGE family, NAG, Tyrosinase, GnT-V, CQA 72/4, Laminin-P1, Yale Col. Sr. Factor, Urinary gonadotropin Peptide (UGP), hCG and chains thereof, BHCG, IL-13Rα2, PD1 (CD279), CTLA-4, PSA, Melan-A/MART-1, gp100, TRP1, MUC-1, MUC-2, beta-catenin, MUM-1, CDK-4, TAG-72, CA-15-3, CA-19-9, CA 72-4, CA-125, Cyfra 21-1, NSE, AMFr, M-344, 19a21 1, erb-2, p15, p21 of ras, mutated p53, Bcr/Abl breakpoint peptide, WT1, HER-2/neu, PD-41, TCSF, GA733-2, HPV16 E7 or E6, MZ2-E, B7.1, B7.2, HOM-MEL-40, HOM-MEL-55, SSX2, NY-ESO-1, SCP1, CT7, NY-COL-2, HOM-HD-397, HOM-RCC-1.14, HOM-HD-21, HOM-NSCLC-11, HOM-MEL-2.4, and HOM-TES-11.

In some embodiments, the tumor antigens of the present disclosure may be selected from AFP, CEA, CD31, CD34, CD99, CD117, GCDFP-15, EMA, ETA, MPG, p97, Neu, c-myc, raf, ras, MAGE, BAGE, DAGE/Prame, GAGE, RAGE SMAGE, NAG, CQA 72/4, Laminin-P1, Yale Col. Sr. Factor, UGP, hCG, PD1 (CD279), PTPRC (CD45), HMB-45, Melan-A/MART-1, Myo D1, MSA, M2-PK, PLAP, PSA, gp100, MUC-1, MUC-2, MUC16, TRP-1, MUM-1, CDK-4, TAG-72, CA-15-3, CA-19-9, CA-27-29, CA-72-4, CA-125, Cyfra 21-1, CYP24, NSE, AMFr, M-344, 19a21 1, erb-2, p15, p21, p53, Bcr/Abl breakpoint peptide, HER-2/neu, PD-41, TCSF, GA733-2, HPV16 E7, E6, MZ2-E, B7.1, B7.2, HOM-MEL-40, HOM-MEL-55, NY-COL-2, HOM-HD-397, HOM-RCC-1.14, HOM-HD-21, HOM-NSCLC-11, HOM-MEL-2.4, HOM-TES-11, GRP78, EGFR, BRCA1, BRCA2, APC, HER2, PSA, NY-ESO-1, 4-5, PSMA, PSCA, EpCam, POA, GnT-V, TERT, calcitonin, calretinin, chromogranin, cytokeratin, desmin, inhibin, keratin, recoverin, kallikrein, beta-catenin, annexin, mammoglobin, tyrosinase, etc.

A tumor antigen may also include one or more genes or DNA sequences encoding the above proteins or peptides. About 400 tumor antigens are currently identified. Periodically updated list of antigens can be found in various databases such as for example in http://cancerimmunity.org/v13p15/ incorporated herein by way of reference. For the purposes of this disclosure any antigen that is not a tumor antigen is considered an alloantigen.

One skilled in the art can identify the presence of a particular tumor by testing for the presence of corresponding tumor markers: e.g., in colorectal cancer: M2-PK, CEA, CA 19-9, CA 125; in breast cancer: CEA, CA 15-3, Cyfra 21-1; in ovary cancer: CEA, CA 19-9, CA 125, AFP, BHCG; in uterine cancer: CEA, CA 19-9, CA 125, Cyfra 21-1, SCC; in prostate cancer: PSA, FPSA, PSCA, PSMA; in testicle cancer: AFP, BHCG, IL-13Rα2; in pancreas/stomach cancer: CEA, CA 19-9, CA 72-4; in liver cancer: CEA, AFP; in esophagus cancer: CEA, Cyfra 21-1; in thyroid cancer: CEA, NSE; in lung cancer: CEA, CA 19-9, CA 125, NSE, Cyfra 21-1; and in bladder cancer: CEA, Cyfra 21-1, and TPA. These tumor markers can also be incorporated into instant composition to serve as tumor antigens.

In some embodiments, the tumor antigen is an antigen derived from a cancer cell, said cancer selected from the group consisting of Adrenal cancer, Anal cancer, Bile Duct cancer, Bladder cancer, Bone cancer, Brain/CNS tumors, Breast cancer, Castleman disease, Cervical cancer, Colon/Rectum cancer, Endometrial cancer, Esophagus cancer, Ewing Tumor, Eye cancer, Gallbladder cancer, Gastric cancer, Gastrointestinal carcinoid tumors, Gastrointestinal Stromal Tumor, Gestational Trophoblastic disease, Hodgkin disease, Kaposi sarcoma, Laryngeal and Hypopharyngeal cancer, Leukemias, including ALL, AML, CLL, CML, and CMML, Lymphoma, Non-Hodgkin lymphoma, Liver cancer, Lung cancer, Malignant mesothelioma, Multiple myeloma, Myelodysplastic syndrome, Nasal cavity and Paranasal sinus cancer, Nasopharyngeal cancer, Neuroblastoma, Oral cavity and Oropharyngeal cancer, Osteosarcoma, Ovarian cancer, Pancreatic cancer, Penile cancer, Pituitary tumor, Prostate cancer, Renal cancer, Retinoblastoma, Rhabdomyosarcoma, Salivary gland cancer, Sarcoma, Basal and squamous cell skin cancer, Melanoma, Merkel cell cancer, Small intestine cancer, Stomach cancer, Testicular cancer, Thymus cancer, Thyroid cancer, Uterine sarcoma, Vaginal cancer, Vulvar cancer, Waldenstrom macroglobulinemia, and Wilms tumor.

Tumor antigens for the present invention can be obtained as a stand-alone antigen, e.g., an isolated and/or purified protein (including a recombinant protein). Alternatively, the tumor antigens can be sourced from surgically resected tumors, body fluids of cancer patients such as blood or ascites, primary tumor cell lines or from immortalized cancer cell lines. Preferably tumor cells are of the same type as cancer in a subject being treated, although this is not critical. One skilled in the art can readily obtain cancer cell lines from numerous cell line depositories or make further cell lines by culturing tumor cells as a primary source. By way of example, ATCC offers a panel of seven liver cancer cell lines which include SNU-387, SNU-423, SNU-449, SNU-475, PLC/PRF/5, SK-HEP-1 and HepG2/C3A. These cells can be used individually or in combination as a source of tumor antigens for the presently disclosed composition. In some embodiments, primary tumor or cancer cells are directly used as a starting material for preparing the present composition without isolating the tumor antigens therefrom.

As used herein the term “allogeneic antigen” or “alloantigen” refers to an antigen which has individual characteristics specific to one person and these are not same in another individual of the same species such as humans for example. Even though each antigen is coded by the same gene, the inter-individual difference is sufficient to trigger the immune response when allogeneic antigen of one individual is in contact with the immune system of another person. Alloantigens are better known in the context of organ transplantation and are roughly divided into minor and major histocompatibility antigens and can cause the grafted tissue rejected. For the purpose of this disclosure the allogeneic antigen can be replaced by or co-exist with a xenogeneic antigen (i.e., antigen from another species) and qualify as being normal, non-malignant antigen as long as they are distinct from the tumor-specific antigens. Xenogeneic antigens may be introduced expressly or may be present in the composition as result of carry-over from cell culture media to which for example bovine or horse sera were added for enhancing cell growth. For the purpose of this disclosure, allogeneic antigens can be sourced from normal hepatocytes; one can use cells offered for example by ScienCell Research Laboratories which have hepatic cell lines such as HHSEC, HH, HHSteC, and HGBF isolated from non-diseased individuals. Another easily accessible source of alloantigens is a peripheral blood.

In some embodiments, the alloantigens can be sourced from the blood of a patient or preferably from a plurality of patients diagnosed with cancer. In certain embodiments, the tumor cells are separated from allogeneic components in the cell suspension during the preparation of the composition. In other embodiments, the tumor cells are not separated from allogeneic cells present in the starting raw preparation. For the purpose of instant invention it is suitable and satisfactory when the weight percent of the tumor cells in the starting raw preparation (before denaturation or hydrolysis) is less than 80%, 50%, 40%, 30%, 20%, 10%, 5% or even lower while the remaining cells are allogeneic cells (i.e., those cells that are not tumor cells).

Using the amount of tumor antigen as a basis, the amount of alloantigen in the present composition can be at least equal by weight to that the tumor antigen, and can also be 2, 3, 5, 10, 20, 30, 50, 100, 200, 300, 500, 1000, 2000, 3000, 5000, 10,000, 20,000, 30,000, 50,000, 100,000, 200,000, 300,000, 500,000, or 1,000,000 times of that of the tumor antigen, or even greater. In preferred embodiments, the ratio between the tumor antigen to alloantigen by weight can be between 1:1 to 1:100,000, or between 1:50 and 1:200. A suitable ratio can be determined by clinical testing using commonly known techniques in the field. As a reference the ratio of circulating tumor cells to non-tumor cells is one that is found in the peripheral blood of a patient with cancer or in pre-cancerous state and is considered to be the optimal ratio. When many starting raw source samples are pooled (e.g., when the source samples are derived from more than one individual), the ratio will be balanced and more closely represent the situation in real-life population. In pooled source the repertoire of antigens will be broader compared to a single source.

In some embodiments, the tumor antigen and the alloantigen of the presently disclosed composition are denatured. As used hereinafter the term “denaturation” is a process in which bioactive macromolecules such as proteins or nucleic acids lose, at least in part, their quaternary structure, tertiary structure, and/or secondary structure in which they exist in their native state, by application of some external stress or reagents. In one embodiment, denaturation is accomplished by applying heat, for example, by heating the tumor antigens and the alloantigens in an autoclave at a temperature of at least at 115° C. for a duration of at least 15 minutes and at an atmospheric pressure of at least 100 kPa (15 psi). These operating parameters (temperature, pressure, duration, etc.) of the autoclave can be made by one of ordinary skill in the art, e.g., by known correlation between these parameters.

In some embodiments, the antigens of the instant composition are hydrolyzed, i.e., their molecular structure is reduced to smaller sized components or fragments, e.g., smaller sized peptides (oligopeptides and polypeptides) as well as free amino acids. The hydrolysis can occur prior to, after, or concurrently with the denaturation.

In preferred embodiments, the hydrolysis is partial, i.e., less than what would be required to reduce given protein(s) entirely to free amino acids. Preferably less than 10% of hydrolyzed antigens consists of free amino acids, more preferably the free amino acid content is less than 8%, and even more advantageously when such content is less than 5%, or less than 3%. In preferred embodiments, the composition after hydrolysis comprises oligopeptides that account for at least 30% by weight of total amount of initial protein. In further embodiments, the oligopeptides can account for at least 35%, at least 40%, at least 45%, at least 50%, at least 55%, or at least 60% by weight of the initial protein.

Examples of hydrolysis reaction useful in the instant invention include acid or alkali hydrolysis, whereby protein is exposed to an acid such as hydrochloric acid or sodium hydroxide.

The degree of hydrolysis and the makeup for the hydrolysis products depend on the hydrolysis reagents used as well as the condition under which the hydrolysis reaction is carried out. For example, a protein such as albumin can be completely hydrolyzed into free amino acids by heating with 6M of hydrochloric acid for about 24 hours at 110° C. By reducing this temperature by half, e.g., 50° C., one may obtain over 80% of initial protein reduced to oligopeptides and free amino acids, the remainder being hydrolyzed into polypeptides. One skilled in the art can readily modify reaction conditions to arrive at desired oligopeptide proportion or desired amino acid length. By way of example, if one reduces hydrochloric acid concentration and/or reduces the reaction temperature, more polypeptides and less free amino acids will yield, and the average chain length of oligopeptides produced will be greater.

Alkaline hydrolysis involves alkali metal hydroxides such as sodium hydroxide (NaOH) or magnesium hydroxide (Mg(OH)₂). For example, albumin hydrolysis at 6M NaOH at 50° C. for 24 h can produce about 80% peptides recovery yield, the rest being free amino acids. The peptides recovered from the hydrolysis can have an average peptide size of about 13 kDa or less, and include oligopeptides having an average chain length of about 12 amino acids. In one example, the hydrolysis products include peptides, over 60% of which larger than 10 kD and about 15% of which between 6 and 10 kDa, and about 1% of which between 1 and 6 kDa. The hydrolysis product also include about 4% of free amino acids, and about 8% of non-soluble protein aggregates. These numbers are by way of example and do not limit one skilled in the art to modify hydrolysis conditions to arrive at different peptide vs. amino acid ratios.

Hydrolysis can also be catalyzed by enzymes—reactions well known to those skilled in the art. Such reactions naturally occur in a human body during digestion of dietary proteins or within cells when processing antigens. For enzyme hydrolysis one selects art-known proteases such as actinidin, aminopeptidase, astacin, carboxypeptidase, caspase, cathepsin, chymosin, chymotrypsin, clostripain, collagenase, elastase, endoproteinase, exopeptidase, kallikrein, metalloproteinase, papain, pepsin, plasmin, pronase, proteinase, renin, serralysin, subtilisin, thermitase, thermolysin, tonin, or trypsin and reduces protein to smaller fragments by established procedures well known to those skilled in the art. For example, typical protease hydrolysis at neutral pH 6.8, temperature 40° C., and incubation period of 8-12 hours can yield about 30% of free amino acids. Accordingly the hydrolysis conditions can be modified to produce desired peptide versus amino acid ratio. The free amino acid contents can be determined using for example L-8900 high-speed amino acid analyzer (Hitachi, Japan).

The metal component of the present invention can be introduced in various ways. Different reactions that can be employed depending on which type of hydrolysis is chosen, e.g., alkaline or acidic hydrolysis. For example, the reaction for a protein undergoing an alkaline hydrolysis or acidic hydrolysis may be illustrated as follows:

Protein(aq)+MgCl₂(aq)+2NaOH(aq)→2NaCl(aq)+Mg(OH)₂(s)+Mg—R(s), or

Protein(aq)+Mg(OH)₂(s)+2HCl(aq)→H₂O(aq)+MgCl₂(aq)+Mg—R(s),

where R may stand for a protein (or peptide) radical, e.g., amide, or a protein (peptide) ion, and (s) stands for solid and (aq) for aqueous or soluble. In the former reaction NaOH is added and in the latter HCl is added—in classical chemistry such type of reaction is known as double replacement or displacement reaction: AB+CD→AD+BC. In the presence of a protein, a metal reacts with the protein and precipitation or aggregation reaction takes place. It is understood that the bonding between the metal and the protein (or peptide) can be covalent in nature, ionic in nature, or have partial characteristics of both. The proportion of reagents in the reaction is generally in equimolar ratio or equal weight/volume ratios, with the ideal proportion easily established without undue experimentation by considering that the highest yield of precipitated antigen is the primary goal.

Although the steps described above are in a sequential order, such processes can also be performed in alternate orders. In other words, any sequence or order of steps disclosed herein does not necessarily indicate a requirement that the steps be performed in that order. The steps of processes described herein may be performed in any order practical. Furthermore, some steps can be performed simultaneously, for example, the hydrolysis step could be carried out at a high temperature, which will then render a separate denaturation step redundant. Another example of simultaneous steps is hydrolysis and precipitation steps, whereby metal bonding to hydrolyzed protein fragments can occur along with protein fragmentation in acid or alkali medium. Yet another example of steps combination is precipitation and drying of the precipitate—this can occur in a vacuum chamber at a high temperature—in such a way precipitation, drying and denaturation processes can take place at the same time. In this situation the hydrolysis step can be the initial step. Regardless of steps by which precipitate is obtained, it is then dried, made into powder and mixed with pharmaceutically acceptable excipients. This mixture is then made into pills, tablets, or capsules (e.g., gelatin capsules enclosing dry powders of active ingredients and excipients). The tablets, pills or capsules may be further coated or otherwise processed by commonly known techniques in the art.

An exemplary composition of a pill or tablet resulting from steps is shown in Table 1 below.

An exemplary composition of a pill or tablet resulting from these steps is shown in Tables 1, 2, 3.

TABLE 1
Elemental Analysis of A Composition According to the
Present Invention

| Element | Weight Percent |
|---|---|
| Aluminum (Al) | 0.0336568% |
| Arsenic (As) | 0.000630125% |
| Boron (B) | 0.282842% |
| Barium (Ba) | 0.000141634% |
| Calcium (Ca) | 0.505907% |
| Cadmium (Cd) | 0.000216786 |
| Chlorine (Cl) | 2.14882% |
| Chromium | 0.00171984% |
| Cooper (Cu) | 0.000812226% |
| Iron (Fe) | 0.141417% |
| Potassium (K) | 0.0222249% |
| Magnesium (Mg) | 95.937% |
| Manganese (Mn) | 0.00430971% |
| Molybdenum (Mo) | 0.0010637% |
| Sodium (Na) | 0.0342262% |
| Nickel (Ni) | 0.00110416% |
| Phosphorus (P) | 0.123825% |
| Sulfur (S) | 0.649338% |
| Silicone (Si) | 0.0929897% |
| Strontium (Sr) | 0.00163023 |
| Titanium (Ti) | 0.00250316% |
| Vanadium (V) | 0.00333273% |
| Zinc (Zn) | 0.00582143% |
| Zirconium (Zr) | 0.00432705% |
| TOTAL | 100% |

In Table 1, the analysis was performed by using inductively coupled plasma atomic emission spectroscopy (ICP-AES) by digesting a composition of the invention with 3 different solvents (HNO₃; HCl+HNO₃; and HNO₃+H₂O₂ solutions). Depending on the solvent used the composition may vary slightly, but the results of all three tests agree substantially.

TABLE 2
Amino acid composition of the antigenic matter (API) of a
composition of the present invention

| Amino acid | Percent |
|---|---|
| Alanine | 7.39% |
| Arginine | 4.08% |
| Aspartic acid | 12.77% |
| Glutamic acid | 15.42% |
| Glycine | 5.13% |
| Histidine | 3.75% |
| Isoleucine | 2.99% |
| Leucine | 10.71% |
| Lysine | 4.84% |
| Methionine | 1.41% |
| Phenylalanine | 5.61% |
| Proline | 5.87% |
| Serine | 6.22% |
| Threonine | 4.99% |
| Tryptophan | 1.11% |
| Tyrosine | 1.57% |
| Valine | 6.14% |
| TOTAL | 100% |

The results presented in Table 2 were obtained by using L-8900 high-speed amino acid analyzer (Hitachi, Japan) by dissolving sample in 0.02N HCl.

TABLE 3
Pharmaceutical excipients of a composition of the present
invention formulated as a pill

| Excipient | weight % |
|---|---|
| Magnesium chloride equivalent | 29.41% |
| Lactose monohydrate | 39.41% |
| Corn Starch | 28.23% |
| Talcum | 1.18% |
| Magnesium Stearate | 0.59% |
| Tablet's Coating | 1.18% |
| Comprising: | |
| Hydroxypropyl methylcellulose | 0.84% |
| Talcum | 0.34% |
| Titanium dioxide | 0.17% |
| Acid Red 18 | 0.17% |
| TOTAL | 100% |

An illustrative embodiment of the preparation method for the present composition is as follows. A pooled sera is obtained from peripheral blood of a plurality of patients with HCC. The sera is hydrolyzed with hydrochloric acid, and then precipitated with 1M magnesium hydroxide, added stepwise until precipitate is formed in the reaction vessel. The precipitate, which contains magnesium bound to alphafetoprotein as the principal tumor antigen and blood alloantigens mixture, among which albumin will be the predominant species, is collected, heat-denatured, reduced to powder and added with standard art-known excipients (see Table 3) to form a pill.

Compositions containing tumor antigens from other cancer types can be prepared in a similar way using sera from pooled peripheral blood of patients having the cancer at issue or tumor tissues obtained by surgical intervention. The vaccine does not have to be particular cancer specific. One can make a broad-spectrum vaccine by having pooled tumor samples or blood from individuals with different cancer types. To facilitate the process and have higher reproducibility tumor cell lines can be used as a source of tumor antigens, for example a broad-spectrum cancer vaccine may comprise breast (MCF7), brain (U87), pancreas (PANC-1), liver (Hep3B), colon (HCT-15), cervical (HeLa), prostate (DU145) and melanoma (MeWo) cancer cell lines mixed at a desired weight ratio. These cell lines can then be combined with sera of pooled peripheral blood and then subject to the hydrolysis, denaturation, and/or metal induced precipitation as described above.

In a further aspect, the present invention provides methods of treating cancer in a subject or patient, and/or preventing or reducing the likelihood of cancer in a subject or patient at risk of developing a cancer, comprising orally administering to the subject an effective amount of the vaccine of the present invention. For purpose of this disclosure, the cancer includes but is not limited to: liver cancer (e.g., hepatoma, cholangiocarcinoma, hepatoblastoma, angiosarcoma, hepatocellular carcinoma, hepatocellular adenoma, hemangioma); lung cancer (e.g., bronchogenic carcinoma, alveolar carcinoma, bronchial adenoma, sarcoma, lymphoma, chondromatous hamartoma, mesothelioma); cardiac cancer (e.g., sarcoma, myxoma, rhabdomyoma, fibroma, lipoma and teratoma); gastrointestinal cancer (e.g., cancers of esophagus, stomach, pancreas, small bowel, and large bowel); genitourinary cancer (e.g., kidney, bladder and urethra, prostate, testis; bone cancer (e.g., osteogenic sarcoma, fibrosarcoma, malignant fibrous histiocytoma, chondrosarcoma, Ewing's sarcoma, malignant lymphoma, multiple myeloma, malignant giant cell tumor chordoma, osteochronfroma, benign chondroma, chondroblastoma, chondromyxofibroma, osteoid osteoma and giant cell tumors); cancers of the nervous system and brain (e.g., of the skull, meninges, brain, and spinal cord); gynecological cancers (e.g., uterus, cervix, ovaries, vulva, vagina); hematologic cancers (e.g., cancers relating to blood, Hodgkin's disease, non-Hodgkin's lymphoma); skin cancers (e.g., malignant melanoma, basal cell carcinoma, squamous cell carcinoma, Karposi's sarcoma, moles dysplastic nevi, lipoma, angioma, dermatofibroma, keloids, psoriasis); and cancers of the adrenal glands (e.g., neuroblastoma).

In some embodiments, the vaccine of the present invention is administered in the subject along with a chemotherapy agent(s), another cancer vaccine or vaccines, and/or immune adjuvant(s). In certain embodiments, the vaccine is given to the subject as a standalone therapy.

In another aspect, the present invention provides methods for treating or ameliorating inflammatory diseases of autoimmune nature (termed also as “autoinflammatory diseases”) where the immune system of the host is directed against its own cells and tissues using the vaccine compositions described herein. A non-limiting list of such conditions includes acute disseminated encephalomyelitis, Addison's disease, agammaglobulinemia, alopecia areata, amyotrophic lateral sclerosis, ankylosing spondylitis, antiphospholipid syndrome, antisynthetase syndrome, atopic allergy, atopic dermatitis, autoimmune aplastic anemia, autoimmune cardiomyopathy, autoimmune enteropathy, autoimmune hemolytic anemia, autoimmune hepatitis, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticaria, autoimmune uveitis, Balo disease, Behçet's disease, Berger's disease, Bickerstaffs encephalitis, Blau syndrome, bullous pemphigoid, cancer, Castleman's disease, celiac disease, Chagas disease, chronic inflammatory demyelinating polyneuropathy, chronic recurrent multifocal osteomyelitis, chronic obstructive pulmonary disease, Churg-Strauss syndrome, cicatricial pemphigoid, Cogan syndrome, cold agglutinin disease, complement component 2 deficiency, contact dermatitis, cranial arteritis, CREST syndrome, Crohn's disease, Cushing's Syndrome, cutaneous leukocytoclastic angiitis, Dego's disease, Dercum's disease, dermatitis herpetiformis, dermatomyositis, diabetes mellitus type 1, diffuse cutaneous systemic sclerosis, Dressler's syndrome, drug-induced lupus, discoid lupus erythematosus, eczema, endometriosis, enthesitis-related arthritis, eosinophilic fasciitis, eosinophilic gastroenteritis, eosinophilic pneumonia, epidermolysis bullosa acquisita, erythema nodosum, erythroblastosis fetalis, essential mixed cryoglobulinemia, Evan's syndrome, fibrodysplasia ossificans progressiva, fibrosing alveolitis, Freiberg's infraction, gastritis, gastrointestinal pemphigoid, glomerulonephritis, Goodpasture's syndrome, Graves' disease, Guillain-Barré syndrome, Hashimoto's encephalopathy, Hashimoto's thyroiditis, Henoch-Schonlein purpura, herpes gestationis, hidradenitis suppurativa, Hughes-Stovin syndrome, hypogammaglobulinemia, idiopathic inflammatory demyelinating diseases, idiopathic pulmonary fibrosis, idiopathic thrombocytopenic purpura, IgA nephropathy, inclusion body myositis, interstitial cystitis, juvenile idiopathic arthritis, Kawasaki's disease, Kienbock's disease, Köhler's disease, Lambert-Eaton myasthenic syndrome, Legg-Calvé-Perthes disease, leukocytoclastic vasculitis, lichen planus, lichen sclerosus, linear IgA disease, lupoid hepatitis, lupus erythematosus, Majeed syndrome, Ménière's disease, microscopic polyangiitis, Miller-Fisher syndromesee Guillain-Barre Syndrome, mixed connective tissue disease, morphea, Mucha-Habermann disease, multiple sclerosis, myasthenia gravis, microscopic colitis, myositis, narcolepsy, neuromyelitis optica, neuromyotonia, occular cicatricial pemphigoid, opsoclonus myoclonus syndrome, Ord's thyroiditis, Osgood-Schlatter's disease, osteochondritis dissecans, palindromic rheumatism, PANDAS, Panner's disease, paraneoplastic cerebellar degeneration, paroxysmal nocturnal hemoglobinuria, Parry Romberg syndrome, Parsonage-Turner syndrome, pars planitis, pemphigus vulgaris, pernicious anaemia, perivenous encephalomyelitis, POEMS syndrome, polyarteritis nodosa, polymyalgia rheumatica, polymyositis, primary biliary cirrhosis, primary sclerosing cholangitis, progressive inflammatory neuropathy, psoriasis, psoriatic arthritis, pyoderma gangrenosum, pure red cell aplasia, Rasmussen's encephalitis, Raynaud phenomenon, relapsing polychondritis, Reiter's syndrome, restless leg syndrome, retroperitoneal fibrosis, rheumatoid arthritis, rheumatic fever, sarcoidosis, schizophrenia, Schmidt syndromeanother form of APS, Scheuermann's disease, Schnitzler syndrome, scleritis, scleroderma, serum sickness, Sever's disease, Sjögren's syndrome, spondyloarthropathy, Still's disease, stiff person syndrome, subacute bacterial endocarditis, Susac's syndrome, Sweet's syndrome, Sydenham chorea, sympathetic ophthalmia, systemic lupus erythematosus, Takayasu's arteritis, temporal arteritis, thrombocytopenia, Tolosa-Hunt syndrome, transverse myelitis, ulcerative colitis, undifferentiated connective tissue disease, undifferentiated spondyloarthropathy, urticarial vasculitis, vasculitis, vitiligo, or Wegener's granulomatosis.

In general, the unit dosage range for both tumor antigens and alloantigens of the invention can be in the order of 0.01 μg-100 mg per individual dose, preferably 0.1 μg-10 mg per dose and more preferably 1 μg-5 mg per dose. For practical purposes and better outcome doses for single intake can be between 10 μg and 1 mg. Preferred tablet sizes for oral administration can be about 0.3 to about 2 gram, about 0.5 to about 1.2 gram, or about 0.8 to about 1 gram. Tablets can be taken one time per day, in other embodiments the frequency is increased up to 4 times per day, the optimal frequency is determined by treating physician based on clinical response.

In some embodiments, the administration of the presently disclosed vaccines is via the enteral route, including the transmucosal delivery of the composition. The method comprises contacting a mucosal surface of the subject in need of such compositions with an effective amount of the composition. Suitable mucosal surfaces include, but are not limited to, oral, buccal, nasal, vaginal, cervical, ocular, auditory, pulmonary tract, urethral digestive tract, skin, mucocutaneous, anal, cloacal, and rectal surface, and the like. In one embodiment, the administration is oral administration, whereby the composition passes the stomach and is absorbed in the intestine very much like any food is absorbed. In other embodiments, the composition is administrated sublingually, rectally, or vaginally.

For oral administration, the presently disclosed vaccine composition can be administered to a subject once per day as a single pill dose. In certain cases dose can be increased to two, three, four pills per day for a duration of at least 14 days, 1 month, 2 months, 3 months, or as long as it is needed. A therapy using the instant composition can last 2 months, 3 months, 6 months, while in some cases as long as one year of continuous dosing may be required.

The composition can be given to a cancer patient regardless whether the disease is in early stage or in terminal stage. The composition can also be given to someone who does not have disease but may be at increased risk or wishes to prevent cancer by taking the vaccine prophylactically.

In certain embodiments, the vaccine of the present invention is administered to the subject as a stand-alone therapy. In other embodiments, the administration of presently disclosed composition can be combined with surgery, radiation, chemotherapy, another immunotherapy, hormone therapy, or any combination thereof. In some embodiments, the vaccine of the present invention is administered in the subject along with a chemotherapy agent(s), another cancer vaccine or vaccines, and/or immune adjuvant(s). The additional therapy can be administered prior to, during, or subsequent to the administration of the presently disclosed composition.

Where the present composition is intended for animal or veterinary use, the composition can be conveniently administered with animal feed, such as grain or food pellets, bait, or in the animal drinking water. The composition may also be incorporated into a food meal, e.g., by spraying it onto the meal.

### EXAMPLES

The invention is further described by the following examples, which do not limit the invention in any manner.

### Example 1: Composition and Preparation

A composition for treating liver cancer was prepared from a pooled sera obtained from peripheral blood of 20 patients with HCC, which was hydrolyzed for about 12 hours at room temperature with 1M of hydrochloric acid and then precipitated with 1M magnesium hydroxide, added stepwise until the precipitate was formed in the reaction vessel. The precipitate, which contains magnesium bound to alphafetoprotein as principal tumor antigen and blood alloantigens mixture, among which albumin will be the predominant species, was collected, heat-denatured in a commercial autoclave for 20 minutes at a temperature of 110° C. and atmospheric pressure of 100 kPa (15 psi), reduced to powder and excipients (as shown in Table 3) were added to form a pill. It is noted that the hydrolysis was partial in the above procedure, with about 80% of the initial antigens broken into peptides of size between 1 kDa and 30 kDa.

The pill prepared according to the above procedure contains a denatured and hydrolyzed alpha-fetoprotein of at least 10 microgram per dose, denatured and hydrolyzed albumin at least 100 microgram per dose, and magnesium metal of at least 200 microgram per dose.

The amino acid makeup of the representative composition so made is shown below. The batch-to-batch variability was within standard deviation range of less than 15% and had no effect on clinical efficacy.

| Amino acid | Percent (%) |
|---|---|
| Ala | 13.1 |
| Arg | 4.5 |
| Asp | 17.2 |
| Cys | 0.7 |
| Glu | 12.7 |
| Gly | 3.2 |
| His | 6.3 |
| Ile | 3.6 |
| Leu | 3.8 |
| Lys | 11.3 |
| Met | 5 |
| Phe | 4.6 |
| Pro | 5.8 |
| Ser | 16 |
| Thr | 10.7 |
| Tyr | 7.9 |
| Val | 11.6 |

The composition above was provided to the HCC patients as discussed in Example 2 below.

The composition provided to patients discussed in Examples 3-13 contains a broad-spectrum cancer vaccine which includes hydrolyzed primary tumor tissues including, where available, their metastases collected by surgical procedure. An equally effective alternative which has been used to treat patients with various tumor types is made from immortalized cancer cell lines grown in vitro. Such a composition contains breast (MCF7), brain (U87), pancreas (PANC-1), liver (Hep3B), colon (HCT-15), cervical (HeLa), prostate (DU145) and melanoma (MeWo) cancer cell lines mixed at equal weight ratio. The cell lines were combined with sera of pooled peripheral blood from healthy individuals and then were subject to the hydrolysis, denaturation, and/or metal induced precipitation as described above.

### Example 2: Liver Cancer

70 patients with advanced HCC, consisting of 26 (37.1%) females and 44 (62.9%) males with median age 60 years (Mean 61.7±8.3) were enrolled to test a vaccine composition of the present invention. Out of these 30 (42.9%) had hepatitis B and 39 (56.5%) hepatitis C infections, 8 (11.4%) with dual infection, 4 (5.7%) negative for both viruses, including 2 with hereditary hemochromatosis (2.9%), and 5 (7.1%) without known diagnosis. After median 2 months of orally taking a daily dose of the vaccine pill, 47 out 70 patients experienced decline in serum levels of tumor marker, alphafetoprotein, (67.1%; P=0.007 by Wilcoxon Signed Rank test). Baseline median AFP levels were 223 IU/ml (Mean 4,328; Range 7.2-92,407; 95% CI 1,077-7,045) and post-treatment values were 101.2 IU/ml (Mean 2,701; Range 0.9-54,478; 95% CI 521-4,881). The decrease in AFP was correlated either with tumor clearance or regression on CT scans. The median overall survival time could not be established since 65 out 70 (92.9%) were still alive after median follow-up of 8 months (Mean 11.2; Range 3-55; 95% CI 8.8-13.5). The first patient in this study received immunotherapy 55 months before and was doing well without any trace of lesions. None of the patients experienced any adverse effects, contrary their liver function tests had improved. The Table 4 shows the outcome of immunotherapy intervention.

TABLE 4
Data for 70 patients with advanced HCC treated with daily dose of a
composition of the instant invention

| | | | | | | | Treatment | |
|---|---|---|---|---|---|---|---|---|
| Patient | | | HBV | HCV | AFP | AFP | duration | Follow-up |
| No | Sex | Age | +/− | +/− | Before | After | (months) | (months) |
| 1 | M | 54 | | + | 428.7 | 2.5 | 12 | 55 |
| 2 | M | 54 | | + | 18.7 | 4.4 | 2 | 47 |
| 3 | F | 58 | | | 1516 | 488.7 | 3 | 29 |
| 4 | M | 58 | + | | 265.1 | 5.5 | 2 | 26 |
| 5 | M | 71 | − | − | 36.7 | 0.9 | 1 | 22 |
| 6 | F | 61 | | + | 57.6 | 22.5 | 4 | 20 |
| 7 | F | 52 | + | | 800 | 100 | 2 | 18 |
| 8 | F | 81 | | + | 7.21 | 69.87 | 12 | 49 |
| 9† | M | 54 | + | + | 13298 | 54478 | 3 | 17 |
| 10 | M | 53 | + | | 1000 | 400 | 3 | 17 |
| 11 | F | 58 | + | + | 39.8 | 8.2 | 3 | 15 |
| 12 | M | 67 | | + | 92407 | 2.15 | 7 | 24 |
| 13 | M | 68 | | + | 1925 | 48.2 | 3 | 17 |
| 14 | F | 51 | | + | 80 | 13.4 | 1 | 13 |
| 15 | F | 65 | | + | 20.32 | 13.97 | 3 | 14 |
| 16 | M | 62 | | + | 16.3 | 2.4 | 1 | 13 |
| 17 | M | 43 | | + | 180.47 | 162.3 | 1 | 12 |
| 18 | F | 60 | + | | 91.3 | 19.2 | 1 | 11 |
| 19 | F | 56 | + | + | 28.4 | 16.4 | 3 | 11 |
| 20 | F | 64 | | + | 47.8 | 32.9 | 5 | 10 |
| 21 | F | 59 | | + | 122.5 | 152.1 | 1 | 11 |
| 22 | M | 60 | | + | 14.95 | 19.51 | 2 | 9 |
| 23 | F | 61 | + | + | 22.54 | 245.5 | 1 | 9 |
| 24 | M | 63 | − | − | 23700.3 | 8.5 | 9 | 10 |
| 25 | M | 59 | + | | 42.92 | 272 | 5 | 8 |
| 26† | M | 58 | | + | 22020 | 21070 | 1 | 7 |
| 27 | M | 62 | | + | 4054 | 4147 | 3 | 7 |
| 28 | F | 50 | + | | 9.26 | 11.48 | 5 | 7 |
| 29 | M | 81 | + | | 12.68 | 41.7 | 5 | 7 |
| 30 | F | 67 | | + | 2750 | 2010 | 1 | 8 |
| 31 | M | 57 | + | + | 476.2 | 41.92 | 7 | 8 |
| 32 | M | 66 | | + | 3188 | 308.1 | 8 | 8 |
| 33 | M | 57 | | + | 320.6 | 117.8 | 2 | 7 |
| 34 | M | 50 | + | | 1000 | 400 | 2 | 6 |
| 35† | M | 72 | | + | 15791 | 175.2 | 2 | 8 |
| 36 | M | 63 | + | + | 9624 | 41.4 | 3 | 12 |
| 37† | M | 52 | + | | 69.35 | 102.3 | 3 | 7 |
| 38 | F | 73 | | | 259.2 | 144 | 1 | 13 |
| 39† | M | 56 | + | | 725 | 1589 | 1 | 9 |
| 40 | M | 80 | | + | 126.8 | 59.4 | 3 | 9 |
| 41 | F | 68 | + | + | 200.7 | 160.1 | 1 | 7 |
| 42 | F | 50 | | | 5768 | 2397 | 2 | 6 |
| 43 | M | 59 | + | | 40 | 33.1 | 2 | 6 |
| 44 | M | 64 | | | 84.4 | 179 | 1 | 8 |
| 45 | M | 58 | + | | 140 | 124 | 1 | 8 |
| 46 | F | 67 | + | | 255.3 | 135.9 | 1 | 7 |
| 47 | M | 72 | + | | 125.5 | 176.6 | 1 | 7 |
| 48 | M | 57 | | | 8 | 12.4 | 1 | 7 |
| 49 | M | 54 | | + | 2100 | 2100 | 2 | 6 |
| 50 | M | 60 | + | | 17.4 | 29.4 | 2 | 7 |
| 51 | F | 68 | | + | 30.1 | 6.96 | 3 | 6 |
| 52 | F | 55 | + | + | 59.46 | 16 | 1 | 8 |
| 53 | M | 61 | + | | 1887 | 4941 | 4 | 10 |
| 54 | F | 70 | + | | 7670 | 5617 | 1 | 5 |
| 55 | F | 60 | + | | 66.55 | 71.1 | 1 | 7 |
| 56 | F | 72 | | + | 436.1 | 1946 | 1 | 6 |
| 57 | M | 48 | | + | 246.4 | 51.9 | 1 | 7 |
| 58 | M | 51 | + | | 2929 | 8750 | 2 | 4 |
| 59 | M | 60 | | + | 14.95 | 22.92 | 2 | 6 |
| 60 | M | 67 | | + | 2750 | 2380 | 1 | 6 |
| 61 | M | 60 | + | | 14.3 | 11.5 | 2 | 4 |
| 62 | M | 69 | − | − | 189.5 | 188.4 | 1 | 3 |
| 63 | M | 57 | | + | 5033.8 | 48.2 | 5 | 7 |
| 64 | M | 69 | | + | 246.8 | 411.1 | 1 | 3 |
| 65 | M | 79 | − | − | 11844 | 16835 | 1 | 3 |
| 66 | M | 59 | | + | 2949 | 6289 | 3 | 5 |
| 67 | F | 79 | | + | 60844 | 48619 | 3 | 5 |
| 68 | F | 68 | | + | 245.2 | 30.1 | 3 | 5 |
| 69 | F | 61 | + | | 163.7 | 112.7 | 3 | 7 |
| 70 | M | 62 | + | | 15.37 | 6.87 | 4 | 7 |
| Median | | 60 | | | 223 | 101.2 | 2 | 8 |
| Mean | | 61.7 | | | 4328 | 2701 | 2.6 | 11.2 |

P = 0.007

Note:
†= patient dead.

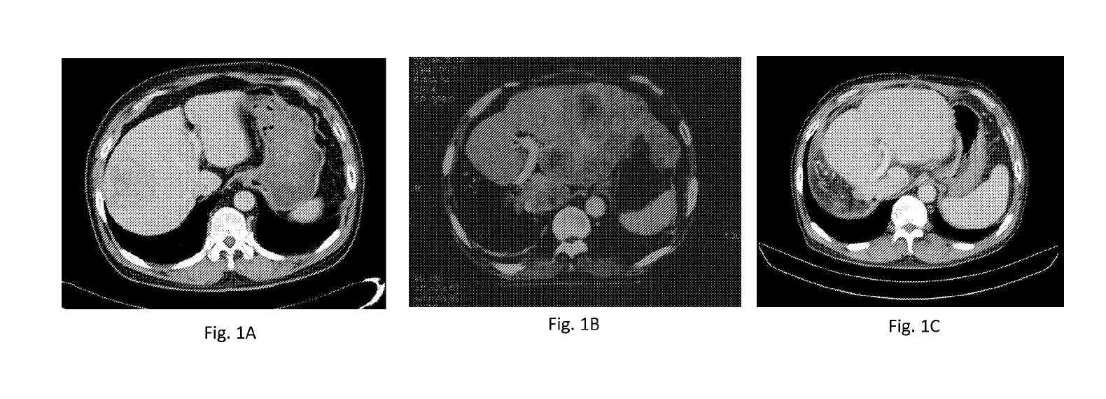

As shown in FIG. 1, CT scans of a representative patient #12 from the above 70 patients before and after daily administration of the composition indicate that multiple tumors in the liver of the patient disappeared without trace after the treatment.

### Example 3: Breast Cancer

Four women with breast cancer diagnosis orally took the instant vaccine once per day. The first two patients had surgery, radiotherapy and chemotherapy, but later on developed metastases, which disappeared without trace after taking vaccine orally once per day for 3 and 6 months, respectively. The third patient had a similar history except that treatment duration was only one month. The fourth lady in her mid-fifties had been diagnosed with breast cancer during routine medical check-up, biopsy finding confirmed the diagnosis. Instead of opting for surgery she decided to take cancer vaccine on “on-off” basis for about one year. After one year she went back to hospital for check-up, her breast cancer markers remained at borderline of normal and she had no detectable tumor mass. No adverse effects were seen in any of four women.

### Example 4: Lung Cancer

One individual in late 50s with terminal non-small cell lung cancer opted to orally take the vaccine of the present invention twice per day as no surgery was possible at this late stage. After 3 months the patient was still alive even though the survival likelihood was one month only. He still apparently had a tumor mass but it appeared reduced in size and he has been able to breathe as usual as fluids which were initially present in his left lung had disappeared.

### Example 5: Esophagus Cancer

A 50-year old male noticed that his voice was getting hoarse and he had difficulties to swallow. The food was getting stuck in the esophagus, he had pain when swallowing, chest pain, heartburn, and had some weight loss. After check-up at the hospital he was diagnosed with esophageal cancer. Instead of surgery and radiotherapy he took the vaccine for 4 months along with some herbal remedies. Patient had no more cancer and went back to work. Another patient who heard of first patient started the treatment but at this stage, one month after treatment, it was unknown what was the outcome except that patient was still alive and had lesser problems with swallowing.

### Example 6: Cervical/Uterine Cancer

Two ladies in their late 40-s have been diagnosed with cervical/uterine cancer. One patient underwent surgery and chemotherapy after which she took the vaccine for 6 months twice per day. One year later she is still alive and well. Second lady took instant vaccine therapy after she started bleeding and lost weight. Hospital declined to admit her since she was inoperable. After taking vaccine for one week she saw that bleeding had stopped, pain in lower abdomen disappeared and after one month she regained weight back to normal. She is still alive as of present time.

### Example 7: Brain Tumor

An HIV-positive patient had experienced progressive head-ache and started having problems with equilibrium when walking, which made him essentially bed-ridden. Radiography discovered large brain tumor that was diagnosed as glioblastoma. After 2 months of taking vaccine once per day of taking the vaccine, the patient regained ability to walk unattended and his head-ache had disappeared as well as tumor mass. Second patient, a female in her 40-s, had been diagnosed with brain tumor which was not amenable by surgery. About 8 months of taking the vaccine she was still taking the vaccine and had no major symptoms except occasional headache. The tumor apparently remained in place but it had not grown in size.

### Example 8: Leukemia

A 13-year old child with acute lymphoid leukemia took the vaccine twice per day and was still alive after 6 months. Another patient in his 30s who was diagnosed with chronic myelogenous leukemia (CML) took the vaccine once per day and was still alive after one year, even though he had no chemotherapy.

### Example 9: Bladder Tumor

An adult male patient diagnosed with bladder tumor took the vaccine for 2 months at 4 pills per day. Apparently his tumor was gone and patient was still alive after one year. Two more patients diagnosed with same cancer type took the vaccine once per day; they were still alive beyond their predicted prognosis of death and symptoms had eased up.

### Example 10: Myosarcoma

A woman in her late 60s was diagnosed with muscle tumor in her legs. She had family history of the disease as her mother and aunt had died from the same disease. She had severe leg and body pain, which disappeared after 2-3 days she started taking the vaccine once per day. After about 3 months she went back to the hospital where doctors were surprised to see her alive and after extensive check-up she was declared cured. The patient returned to her normal activities including daily bicycle rides and ballroom dance exercises.

### Example 11: Bone Cancer

A 75-year old male patient had osteosarcoma without apparent metastases for about 3 years and was bedridden most of the day. He started taking the vaccine once per day for 3 months, and his condition had improved to the extent that he could walk and his demand for pain-killers has been reduced considerably. He gained weight and his urine incontinence had stopped.

### Example 12: Prostate Cancer

Two males aged 56 and 62 who had history of benign prostatic hyperplasia were suspected to have malignant transformation due to pelvic and vertebrae pain and higher than normal PSA findings. Instead of opting for surgery they took daily dose of the vaccine for about two months and their symptoms disappeared. Their baseline urinary frequency, incomplete bladder emptying and hesitancy dissipated and returned to normal levels prior to BHP diagnosis.

### Example 13: Measuring Immune Response Induced by Instant Vaccine

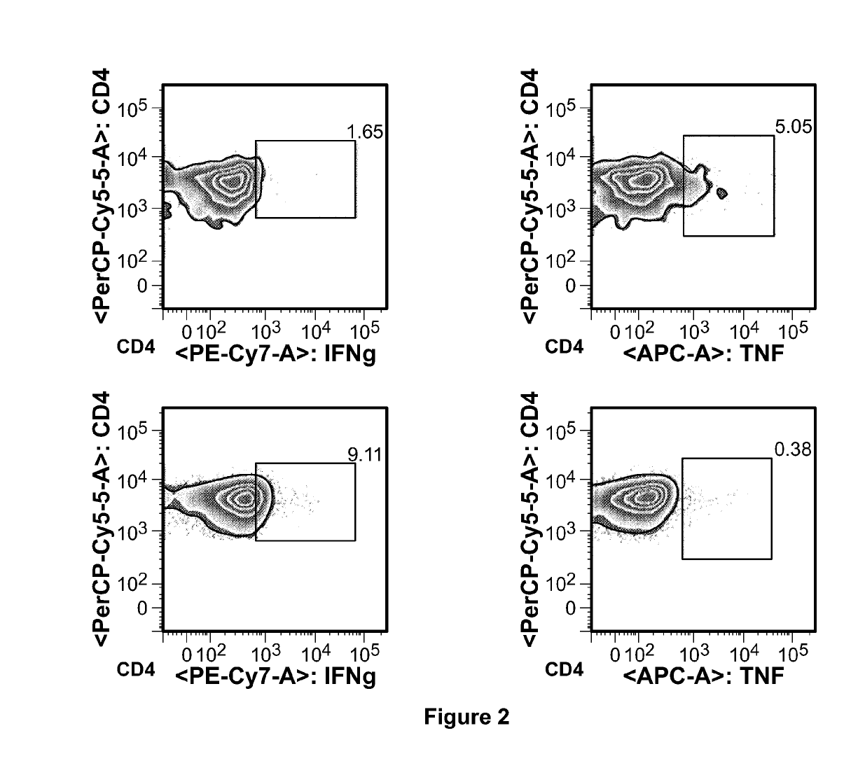

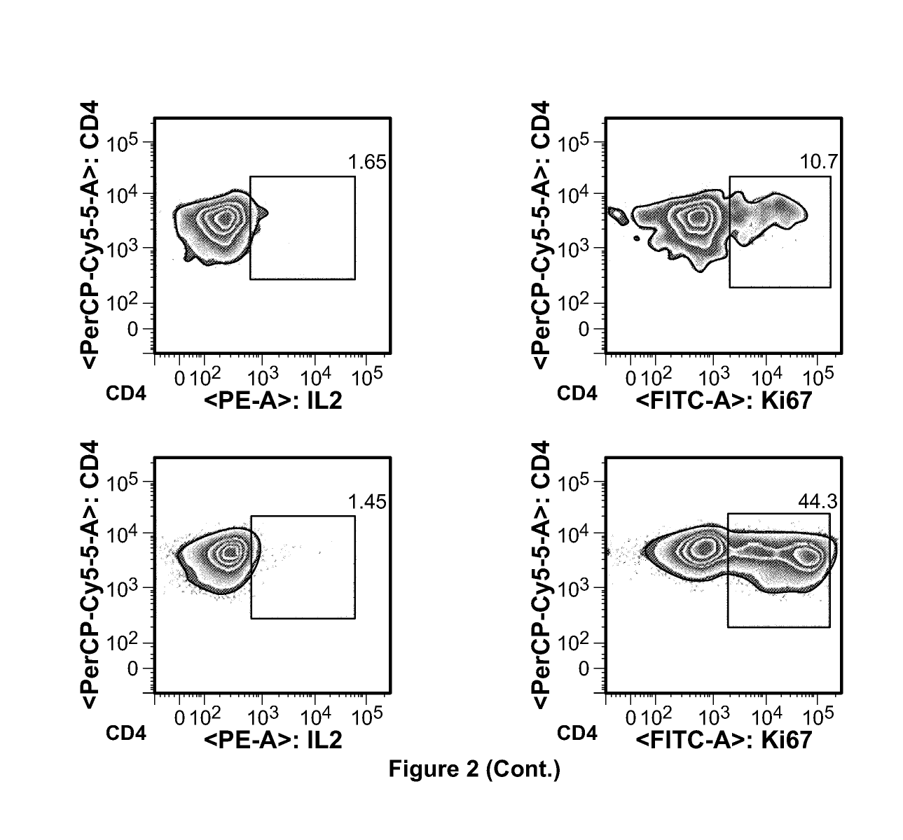

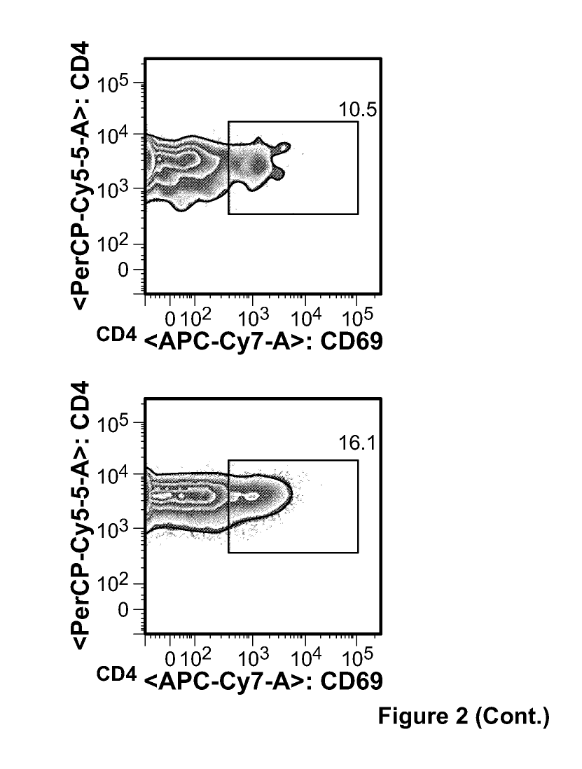

Freshly isolated peripheral blood cells were incubated with a vaccine of the present invention diluted a million-fold (1 ppm or 1:10⁻⁶) for 48 hours. This dilution represents the approximate physiological dose of composition after it is taken orally. Flow cytometry on in vitro grown cells was then performed on CD4+positive population of T lymphocytes to assess the effect of instant composition on select markers of immune activation in vaccine exposed cells as compared with control cells which were not exposed to the vaccine. Results indicate that cells have increased expression of IFN-gamma by almost 6-fold or 600%; decrease by 13-fold (1,300%) of cells with inflammation marker TNF-alpha, essentially had no effect on IL-2 expression; increased CD69 expression which is the marker of T-cell activation, also increased the frequency of Ki-67 cells, which is the marker of vaccine-induced proliferating memory cells, by 4-fold (400%). Similar results were seen with CD8-positive T cells. Thus, the physiological concentration of the vaccine had very strong and fast effect on immune cells. Such an anti-inflammatory effect is unique and has never been reported with other cancer vaccines (see for example PCT/US2012/024009 in which the highest increase of Ki-67 was 95% after 29 days). The pattern of change of biomarkers suggests that the instant vaccine has a potent anti-inflammatory effect as shown in FIG. 2. These in vitro assays are not limited to flow cytometry, one skilled in the art can use other in vitro assays such as ELISA method, mRNA expression of cytokines assay or multiplex assay, described for example in U.S. Pat. No. 5,223,395, EP1664796 B1 and EP1222468 B1 which are incorporated herein by reference.

It is to be understood that while the invention has been described in detail by way of example and illustration for the purpose of clarity of teaching, the foregoing description is not intended to limit the scope of the invention. Other aspects, advantages, and modifications that are apparent to one of skill in the art are within the scope of the following claims.

What is claimed is:

1. An oral composition comprising a metal bound to at least one tumor antigen and at least one alloantigen or fragments thereof.

2. The composition of claim 1, wherein said metal is magnesium or calcium.

3. The composition of claim 1, wherein at least one tumor antigen and at least one alloantigen is hydrolyzed.

4. The composition of claim 1, wherein the tumor antigen is selected from a group consisting of a protein, a peptide, a hapten, a polysaccharide, a glycoprotein, a lipopolysaccharide, and a DNA molecule.

5. The composition of claim 1, wherein at least one tumor antigen and at least one alloantigen is heat-denatured.

6. The composition of claim 4, wherein the protein is AFP.

7. The composition of claim 1, wherein the tumor antigen is selected from the group consisting of AFP, CEA, CD31, CD34, CD99, CD117, GCDFP-15, EMA, ETA, MPG, p97, Neu, c-myc, raf, ras, MAGE, BAGE, DAGE/Prame, GAGE, RAGE SMAGE, NAG, CQA 72/4, Laminin-P1, Yale Col. Sr. Factor, UGP, hCG, PD1 (CD279), PTPRC (CD45), HMB-45, MART-1/Melan-A, Myo D1, MSA, M2-PK, PLAP, PSA, gp100, MUC-1, MUC-2, MUC16, TRP-1, MUM-1, CDK-4, TAG-72, CA-15-3, CA-19-9, CA-27-29, CA-72-4, CA-125, Cyfra 21-1, CYP24, NSE, AMFr, M-344, 19a21 1, erb-2, p15, p21, p53, Bcr/Abl breakpoint peptide, WT1, HER-2/neu, PD-41, TCSF, GA733-2, HPV16 E7, E6, MZ2-E, B7.1, B7.2, HOM-MEL-40, HOM-MEL-55, NY-COL-2, HOM-HD-397, HOM-RCC-1.14, HOM-HD-21, HOM-NSCLC-11, HOM-MEL-2.4, HOM-TES-11, GRP78, EGFR, BRCA1, BRCA2, APC, HER2, PSA, NY-ESO-1, 4-5, PSMA, PSCA, EpCam, POA, GnT-V, TERT, calcitonin, calretinin, chromogranin, cytokeratin, desmin, inhibin, keratin, recoverin, kallikrein, beta-catenin, annexin, mammoglobin, tyrosinase and mixtures thereof.

8. The composition of claim 1, wherein the tumor antigen is an antigen derived from a cancer cell, said cancer selected from the group consisting of Adrenal cancer, Anal cancer, Bile Duct cancer, Bladder cancer, Bone cancer, Brain/CNS tumors, Breast cancer, Castleman disease, Cervical cancer, Colon/Rectum cancer, Endometrial cancer, Esophagus cancer, Ewing Tumor, Eye cancer, Gallbladder cancer, Gastric cancer, Gastrointestinal carcinoid tumors, Gastrointestinal Stromal Tumor, Gestational Trophoblastic disease, Hodgkin disease, Kaposi sarcoma, Laryngeal and Hypopharyngeal cancer, Leukemias, including ALL, AML, CLL, CML, and CMML, Lymphoma, Non-Hodgkin lymphoma, Liver cancer, Lung cancer, Malignant mesothelioma, Multiple myeloma, Myelodysplastic syndrome, Nasal cavity and Paranasal sinus cancer, Nasopharyngeal cancer, Neuroblastoma, Oral cavity and Oropharyngeal cancer, Osteosarcoma, Ovarian cancer, Pancreatic cancer, Penile cancer, Pituitary tumor, Prostate cancer, Renal cancer, Retinoblastoma, Rhabdomyosarcoma, Salivary gland cancer, Sarcoma, Basal and squamous cell skin cancer, Melanoma, Merkel cell cancer, Small intestine cancer, Stomach cancer, Testicular cancer, Thymus cancer, Thyroid cancer, Uterine sarcoma, Vaginal cancer, Vulvar cancer, Waldenstrom macroglobulinemia, and Wilms tumor.

9. The composition of claim 1, wherein the alloantigen is albumin.

10. The composition of claim 1, wherein the weight ratio between the tumor antigen and the alloantigen is between 1:1 to 1:100,000, or between 1:50 and 1:200.

11. A composition comprising a metal bound to at least one heat-denatured, hydrolyzed alloantigen and at least one heat-denatured, hydrolyzed tumor antigen, said composition formulated as a pill.

12. The composition of claim 11, wherein said metal is magnesium or calcium.

13. The composition of claim 11, wherein said hydrolyzed and denatured tumor antigen and hydrolyzed and denatured alloantigen is a peptide.

14. The composition of claim 13, wherein said peptide is an oligopeptide.

15. The composition of claim 13, wherein said peptide is a polypeptide.

16. A method for treating a cancer in a subject in need thereof, comprising orally administering to the subject a therapeutically effective dose of a composition according to any of claims 1-15.

17. A method of inducing an anti-inflammatory immune reaction in a subject, the method comprising orally administering a therapeutically effective dose of a composition to the subject, the composition comprising a tumor antigen and an alloantigen bound to a metal.

18. A method of claim 17 wherein the tumor antigen and alloantigen are hydrolyzed.

19. A method of claim 17 wherein the tumor antigen and alloantigen are heat-denatured.

20. A method of preparing a composition, comprising:
obtaining a mixture of a tumor antigen and an alloantigen;
denaturing the tumor antigen and the alloantigen;
forming at least one of a metal-bound denatured tumor antigen and a metal-bound denatured alloantigen.

21. The method of claim 20, further comprising:
hydrolyzing at least one of the tumor antigen and the alloantigen.

22. The method of claim 20, wherein the tumor antigen is obtained from a pooled blood of donors diagnosed with desired types of cancer.

23. The method of claim 20, wherein the tumor antigen is obtained from cancer cell lines or cancer tissues.

24. The method of claim 20, wherein the alloantigen is derived from non-malignant cells derived from the peripheral blood or cell lines.

25. The method of claim 20, wherein the denaturing step comprises applying heat.
